SEQUENCE LISTING (13 sequences)

SEQ ID NO: 1 - protein, 76 aa, synthetic construct
  REGION 1..76: note = Artificial Sequence (224039-2)
  VARIANT 6: note = F may be Y or W
  VARIANT 8: note = D may be E, K, R or H
  VARIANT 62: note = W may be Q or A
MQIFVFTDTGKTITLEVEPSDTIENVKAKIQDKEGIPPDQQRLIWAGKQLEDGRTLSDYNIWPRRLLHLVLRLRAA

SEQ ID NO: 2 - protein, 76 aa, synthetic construct
  REGION 1..76: note = Artificial Sequence (224058-2)
  VARIANT 6: note = F may be Y or W
  VARIANT 8: note = D may be E, K, R or H
  VARIANT 62: note = Q may be W or A
MQIFVFTDTGKTITLEVEPSDTIENVKAKIQDKEGIPPDQQRLIWAGKQLEDGRTLSDYNIQPRRLLHLVLRLRAA

SEQ ID NO: 3 - protein, 76 aa, synthetic construct
  REGION 1..76: note = Artificial Sequence (224180-2)
  VARIANT 6: note = F may be Y or W
  VARIANT 8: note = D may be E, K, R or H
  VARIANT 62: note = A may be W or Q
MQIFVFTDTGKTITLEVEPSDTIENVKAKIQDKEGIPPDQQRLIWAGKQLEDGRTLSDYNIAPRRLLHLVLRLRAA

SEQ ID NO: 4 - protein, 76 aa, synthetic construct
  REGION 1..76: note = Artificial Sequence (224039-1/224058-1/224180-1/224121-1/211828-1)
MQIFVDTLTGKTITLEVEPSDTIENVKAKIQDKEGIPPDQQRLIWAGKQLEDGRTLSDYNIRYPAFLHLVLRLRAA

SEQ ID NO: 5 - protein, 152 aa, synthetic construct
  REGION 1..152: note = Artificial Sequence (224039)
MQIFVDTLTGKTITLEVEPSDTIENVKAKIQDKEGIPPDQQRLIWAGKQLEDGRTLSDYNIRYPAFLHLVLRLRAAMQIFVFTDTGKTITLEVEPSDTIENVKAKIQDKEGIPPDQQRLIWAGKQLEDGRTLSDYNIWPRRLLHLVLRLRAA

SEQ ID NO: 6 - protein, 152 aa, synthetic construct
  REGION 1..152: note = Artificial Sequence (224058)
MQIFVDTLTGKTITLEVEPSDTIENVKAKIQDKEGIPPDQQRLIWAGKQLEDGRTLSDYNIRYPAFLHLVLRLRAAMQIFVFTDTGKTITLEVEPSDTIENVKAKIQDKEGIPPDQQRLIWAGKQLEDGRTLSDYNIQPRRLLHLVLRLRAA

SEQ ID NO: 7 - protein, 162 aa, synthetic construct
  REGION 1..152: note = Artificial Sequence (224180)
MQIFVDTLTGKTITLEVEPSDTIENVKAKIQDKEGIPPDQQRLIWAGKQLEDGRTLSDYNIRYPAFLHLVLRLRAAMQIFVFTDTGKTITLEVEPSDTIENVKAKIQDKEGIPPDQQRLIWAGKQLEDGRTLSDYNIAPRRLLHLVLRLRAASAWSHPQFEK

SEQ ID NO: 8 - protein, 152 aa, synthetic construct
  REGION 1..152: note = Artificial Sequence (224121)
MQIFVDTLTGKTITLEVEPSDTIENVKAKIQDKEGIPPDQQRLIWAGKQLEDGRTLSDYNIRYPAFLHLVLRLRAAMQIFVHTATGKTITLEVEPSDTIENVKAKIQDKEGIPPDQQRLIWAGKQLEDGRTLSDYNIWPRRLLHLVLRLRAA

SEQ ID NO: 9 - protein, 152 aa, synthetic construct
  REGION 1..152: note = Artificial Sequence (211828)
MQIFVDTLTGKTITLEVEPSDTIENVKAKIQDKEGIPPDQQRLIWAGKQLEDGRTLSDYNIRYPAFLHLVLRLRAAMQIFVRTTTGKTITLEVEPSDTIENVKAKIQDKEGIPPDQQRLIWAGKQLEDGRTLSDYNIWPRRLLHLVLRLRAA

SEQ ID NO: 10 - protein, 76 aa, synthetic construct
  REGION 1..76: note = Ubiquitin
MQIFVKTLTGKTITLEVEPSDTIENVKAKIQDKEGIPPDQQRLIFAGKQLEDGRTLSDYNIQKESTLHLVLRLRAA

SEQ ID NO: 11 - protein, 220 aa, synthetic construct
  REGION 1..220: note = Extracellular domain of hPD-L1
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 12 - protein, 76 aa, synthetic construct
  REGION 1..76: note = Artificial Sequence (224121-2)
MQIFVHTATGKTITLEVEPSDTIENVKAKIQDKEGIPPDQQRLIWAGKQLEDGRTLSDYNIWPRRLLHLVLRLRAA

SEQ ID NO: 13 - protein, 76 aa, synthetic construct
  REGION 1..76: note = Artificial Sequence (211828-2)
MQIFVRTTTGKTITLEVEPSDTIENVKAKIQDKEGIPPDQQRLIWAGKQLEDGRTLSDYNIWPRRLLHLVLRLRAA

(19) United States
(12) Patent Application Publication
(10) Pub. No.: US 2026/0159588 A1
(43) Pub. Date: Jun. 11, 2026

(54) NOVEL PROTEINS WITH HIGH BINDING AFFINITY TO PROGRAMMED DEATH LIGAND 1 (PD-L1)

(71) Applicant: Navigo Proteins GmbH, Halle/Saale (DE)

(72) Inventors: Jonathan Lotze, Halle/Saale (DE); Ina Coburger, Halle/Saale (DE); Manja Gloser-Braeunig, Halle/Saale (DE); Hanna Bobolowski, Halle/Saale (DE)

(73) Assignee: Navigo Proteins GmbH, Halle/Saale (DE)

(21) Appl. No.: 19/124,117

(22) PCT Filed: Oct. 23, 2023
(86) PCT No.: PCT/EP2023/079483

§ 371 Date: Apr. 24, 2025

(30) Foreign Application Priority Data
Oct. 24, 2022 (EP) 22203393.8

(51) Int. Cl. C07K 16/28; G01N 33/575

(52) U.S. Cl. CPC C07K 16/2827; G01N 33/5758; C07K 2317/52; C07K 2317/92; C07K 2317/94; G01N 2333/70532

(57) ABSTRACT

The present invention relates to new proteins that are specific for Programmed (Cell) Death Ligand 1 (PD-L1). The PD-L1 specific proteins of the invention bind with high affinity to human and mouse PD-L1. The invention further refers to PD-L1 specific proteins that further comprise a diagnostically or therapeutically active component. Further aspects of the invention cover the use of these PD-L1 specific binding proteins in medicine, for example, in diagnosis and therapy of PD-L1 related cancer.

### FIELD OF THE INVENTION

The present invention relates to new proteins that are specific for Programmed (Cell) Death Ligand 1 (PD-L1). The PD-L1 specific proteins of the invention bind with high affinity to human and mouse PD-L1. The invention further refers to PD-L1 specific proteins that further comprise a diagnostically or therapeutically active component. Further aspects of the invention cover the use of these PD-L1 specific binding proteins in medicine, for example, in diagnosis and therapy of PD-L1 related cancer.

### BACKGROUND OF THE INVENTION

The transmembrane protein Programmed (Cell) Death Ligand 1 (PD-L1; also known as B7-H, B7H1, CD274) belongs to the B7 family of immune regulatory molecules and is involved in the regulation of cellular and humoral immune responses. The PD-L1 ligand interacts with PD-1 (programmed cell death 1). PD-L1 is expressed on a variety of normal and immune cells, and tumor cells express PD-L1 as a mechanism of immune evasion.

Both PD-1 and PD-L1 belong to the family of immune checkpoint proteins that act as co-inhibitory factors. They can halt or limit the development of the T cell response, for example suppress T cell activation and proliferation and induce the apoptosis of activated T cells. The PD-1/PD-L1 interaction ensures that the immune system is activated only at the appropriate time in order to minimize the possibility of chronic autoimmune inflammation.

PD-L1 expressed on tumor cells binds to the PD-1 receptor on activated T cells, which leads to the inhibition of cytotoxic T cells and thereby to reduced cytokine production and proliferation of T cells. Through up-regulation of PD-L1 expression, tumor cells escape detection and destruction by the immune system. In cancer, PD-L1 provides resistance to T cell mediated cytotoxicity. By blocking immune checkpoint receptor PD-1 or its ligand PD-L1, the immune system can overcome the cancer's ability to resist the immune responses and stimulate the body's own mechanisms to remain effective in its defenses against cancer.

Several immune checkpoint humanized IgG₁ antibodies were developed as PD-1/PD-L1 inhibitors for cancer immunotherapy, for example, Atezolizumab, Durvalumab, Avelumab, BMS-936559, MEDI4736.

Diagnosis and treatment of PD-L1 related cancer is often not adequately addressed by existing options, and as a consequence, many patients do not adequately benefit from current strategies.

Needless to say that there is a strong need for novel strategies for diagnosis and treatment of tumors with PD-L1 overexpression, as well as other PD-L1 related diseases and disorders, including (viral) infections.

One objective of the present invention is the provision of proteins for specific targeting of PD-L1 for allowing targeted diagnostic and treatment options, including detection of PD-L1 positive tumors (e.g. by radio-diagnostic methods). Targeting this tumor-associated protein may offer benefit to patients with unmet need for novel diagnostic and therapeutic routes. Targeting PD-L1 suggests a potentially non-toxic diagnostic and treatment approach, due to low and restricted distribution of PD-L1 in normal tissues. Thus, binding proteins with specificity for PD-L1 may enable effective medical options for cancer treatment, and finally improve quality of life for patients.

The invention provides novel PD-L1 binding molecules for new and improved strategies in the diagnosis and treatment of PD-L1 positive tumors. Further, the novel PD-L1 binding molecules of the invention provide improved strategies in the diagnosis and treatment of infectious diseases such as chronic viral infection.

The above-described objectives and advantages are achieved by the subject-matter of the appended claims. The present invention meets the needs presented above by providing novel high affinity PD-L1 binding proteins. The above overview does not necessarily describe all problems solved by the present invention.

### SUMMARY OF THE INVENTION

The present disclosure provides the following items 1 to 15, without being specifically limited thereto:
1. A protein comprising an amino acid sequence with at least 90% identity to the amino acid sequence of SEQ ID NO: 1, wherein the protein: (i) has an aromatic amino acid selected from phenylalanine (F), tyrosine (Y), or tryptophan (W) at the position corresponding to position 6 of SEQ ID NO: 1, and an acidic amino acid selected from aspartic acid (D) or glutamic acid (E) at the position corresponding to position 8 of SEQ ID NO: 1; and (ii) exhibits a binding affinity for human Programmed (Cell) Death Ligand 1 (PD-L1) of less than 100 nM; preferred aspects relate to a dimeric PD-L1 binding protein comprising the amino acid sequence of SEQ ID NO: 4 and an amino acid sequence with at least 96% identity to the amino acid sequence of SEQ ID NO: 1, wherein the protein: (i) has an aromatic amino acid selected from phenylalanine (F), tyrosine (Y), or tryptophan (W) at the position corresponding to position 6 of SEQ ID NO: 1, and an acidic amino acid selected from aspartic acid (D) or glutamic acid (E) at the position corresponding to position 8 of SEQ ID NO: 1; and (ii) exhibits a binding affinity for human Programmed Death Ligand 1 (PD-L1) of less than 15 nM as determined by Surface Plasmon Resonance.
2. The protein according to item 1, wherein the protein has a phenylalanine (F) at the position corresponding to position 6 of SEQ ID NO: 1, and an aspartic acid (D) at the position corresponding to position 8 of SEQ ID NO: 1.
3. The protein according to item 1 or 2, wherein protein is stable in serum after 24 h incubation at 37° C.
4. The protein according to any one of items 1 to 3, wherein the protein exhibits a binding affinity for human PD-L1 of less than 15 nM.
5. A multimer comprising the protein according to any one of items 1 to 4, wherein the multimer is a dimer, a trimer, a tetramer, a pentamer, or a hexamer.
6. The multimer according to item 5, comprising the protein according to any one of items 1 to 4 and a protein comprising an amino acid sequence with at least 90% identity to the amino acid sequence of SEQ ID NO: 4, wherein the multimer is preferably a dimer.
7. The protein according to any one of items 1 to 6, further comprising one or more coupling sites for the coupling of chemical moieties, wherein the chemical moieties are preferably selected from any one of chelators, drugs, toxins, dyes, and small molecules.
8. The protein according to any one of items 1 to 7, further comprising at least one diagnostically active moiety, or at least one therapeutically active moiety.
9. The protein according to any one of items 1 to 8, further comprising at least one moiety modulating pharmacokinetics, wherein the at least one moiety modulating pharmacokinetics is preferably selected from any one of a serum albumin; an albumin binding protein; an immunoglobulin binding protein; an immunoglobulin or immunoglobulin fragment; a polysaccharide; an unstructured amino acid sequence comprising amino acids alanine, glycine, serine, proline; a polyethylene glycol; a sialic acid; a transferrin; and a transferrin receptor binding protein; or any combination thereof.
10. The protein according to any one of items 1 to 9 for use in diagnosis or treatment of PD-L1 positive tumors or for use in diagnosis or treatment of infectious diseases such as chronic viral infection.
11. A composition comprising the protein according to any one of items 1 to 10.
12. The composition of item 11 for use in medicine.
13. The composition for use in medicine according to item 12, which is for use in the diagnosis or treatment of PD-L1 positive tumors or for use in the diagnosis or treatment of infectious diseases such as chronic viral infection.
14. A method of producing the protein according to any one of items 1 to 10, comprising the steps of a) culturing a host cell under conditions suitable to obtain said protein and b) isolating said protein produced.
15. A method of detecting PD-L1 in a sample, the method comprising detecting the binding of PD-L1 with a protein according to any one of items 1 to 10 in the sample by contacting the sample with a protein according to any one of items 1 to 10.

This summary does not necessarily describe all features of the present invention. Other embodiments come apparent from a review of the ensuing detailed description.

### BRIEF DESCRIPTION OF THE FIGURES

The FIGURES show:

FIG. 1: shows the binding affinity of dimeric PD-L1 binding proteins after long-term incubation in human serum. After 24 h incubation in serum, the binding affinity (K_{D} value) to PD-L1 expressed on cells showed only minor variation. The binding affinity of 224058 (comprising SEQ ID NO: 2) to PD-L1 after incubation in human serum is shown in FIG. 1A. The binding affinity of 224180 (comprising SEQ ID NO: 3) to PD-L1 after incubation in human serum is shown in FIG. 1B. The results confirm the stability of PD-L1 binding proteins. Even after long term incubation in human serum, the PD-L1 binding proteins bind with high affinity to PD-L1 expressed on cells.

### DETAILED DESCRIPTION OF THE INVENTION

The present inventors have developed a solution to meet the strong ongoing need in the art for expanding medical options for the diagnosis and treatment of cancer and infectious diseases by providing novel PD-L1 binding proteins. The PD-L1 specific proteins as defined herein are functionally characterized by high specific affinity for (human) PD-L1 of less than 100 nM, preferably less than 15 nM. Further, they show high levels of stability, both in serum and at high temperatures. The PD-L1 binding proteins of the invention, including multimeric forms thereof disclosed herein, may inhibit the interaction of PD-L1 with PD-1. This inhibition is considered to provide for blocking the PD-1 inhibitory signalling. The PD-L1 binding proteins as described herein thereby provide molecular formats with favorable physicochemical properties, high-level expression in bacteria, and allow easy production methods. The novel proteins as described herein may broaden so far unmet medical strategies for the diagnosis and therapy of PD-L1 related cancer and infectious diseases. In particular, the protein as described herein may be used for imaging purposes, for example, for the presence of tumor cells expressing PD-L1, and for radiotherapy treatment of tumors expressing PD-L1, or for immunooncological treatment options.

Before the present invention is described in more detail below, it is to be understood that this invention is not limited to the particular methodology, protocols and reagents described herein as these may vary. It is also to be understood that the terminology used herein is for the purpose of describing particular aspects and embodiments only and is not intended to limit the scope of the present invention which is reflected by the appended claims. Unless defined otherwise, all technical and scientific terms used herein have the same meaning as commonly understood by one of ordinary skill in the art to which this invention belongs. This includes a skilled person working in the field of protein engineering and purification, but also including a skilled person working in the field of developing new target-specific binding molecules for use in technical applications and in therapy and diagnostics.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Throughout this specification and the claims, which follow, unless the context requires otherwise, the word “comprise”, and variants such as “comprises” and “comprising”, was understood to imply the inclusion of a stated integer or step, or group of integers or steps, but not the exclusion of any other integer or step or group of integers or steps. The term “comprise(s)” or “comprising” may encompass a limitation to “consists of” or “consisting of”, should such a limitation be necessary for any reason and to any extent.

Several documents (for example: patents, patent applications, scientific publications, manufacturer's specifications, instructions, GenBank Accession Number sequence submissions etc.) may be cited throughout the present specification. Nothing herein is to be construed as an admission that the invention is not entitled to antedate such disclosure by virtue of prior invention. Some of the documents cited herein may be characterized as being “incorporated by reference”. In the event of a conflict between the definitions or teachings of such incorporated references and definitions or teachings recited in the present specification, the text of the present specification takes precedence.

All sequences referred to herein are disclosed in the attached sequence listing that, with its whole content and disclosure, forms part of the disclosure content of the present specification.

### General Definitions of Important Terms Used in the Application

The term “PD-L1” as used herein refers to Uniprot accession number Q9NZQ7 (programmed cell death ligand 1, or programmed death ligand 1). The term “PD-L1” comprises all polypeptides which show a sequence identity of at least 70%, 75%, 80%, 85%, 90%, 95%, 96% or 97% or more, or 100% to the PD-L1 of Uniprot accession number Q9NZQ7 (human). The human PD-L1 is 73% identical zu mouse PD-L1 (accession number Q9EP73) and 74% identical to rat PD-L1. The term “PD-L1” includes the extracellular domain (residues 19-238) of PD-L1. Human PD-L1 possesses three domains: an extracellular domain (aa 19-238), a transmembrane domain (aa 239-259), and a cytoplasmic domain (aa 260-290). The extracellular domain of human PD-L1 is shown in SEQ ID NO: 11.

The term “PD-L1 binding protein” refers to a protein of the present invention, including multimeric forms thereof disclosed herein, with high affinity binding to PD-L1 (programmed cell death ligand 1, or programmed death ligand 1). Accordingly, the proteins of the present disclosure are PD-L1 binding proteins. As described elsewhere herein, the proteins of the present disclosure exhibit a specific binding affinity for human PD-L1, which is a binding affinity in the nanomolar range.

The terms “protein” and “polypeptide” refer to any chain of two or more amino acids linked by peptide bonds, and does not refer to a specific length of the product. Thus, “peptides”, “protein”, “amino acid chain”, or any other term used to refer to a chain of two or more amino acids, are included within the definition of “polypeptide”, and the term “polypeptide” may be used instead of, or interchangeably with, any of these terms. The term “polypeptide” is also intended to refer to the products of post-translational modifications of the polypeptide, which are well known in the art.

The term “modification” or “amino acid modification” refers to a substitution, a deletion, or an insertion of a reference amino acid at a particular position in a parent polypeptide sequence by another amino acid. Given the known genetic code, and recombinant and synthetic DNA techniques, the skilled scientist can readily construct DNAs encoding the amino acid variants.

The term “amino acid substitution” is understood as an exchange of an amino acid by another amino acid.

The term “ubiquitin” refers to the amino acid sequence given in SEQ ID NO: 10.

The terms “binding affinity” and “binding activity” may be used herein interchangeably, and they refer to the ability of a polypeptide to bind to another protein, peptide, or fragment or domain thereof. Binding affinity is typically measured and reported by the equilibrium dissociation constant (K_{D}), which is used to evaluate and rank order strengths of biomolecular interactions.

The term “fusion protein” relates to a protein comprising at least a first protein joined genetically to at least a second protein. A fusion protein is created through joining of two or more genes that originally coded for separate proteins. Fusion proteins may further comprise additional domains that are not involved in binding of the target, such as but not limited to, for example, multimerization moieties, polypeptide tags, polypeptide linkers or moieties binding to a target different from PD-L1.

The term “amino acid sequence identity” refers to a quantitative comparison of the identity (or differences) of the amino acid sequences of two or more proteins. “Percent (%) amino acid sequence identity” with respect to a reference polypeptide sequence is defined as the percentage of amino acid residues in a sequence that are identical with the amino acid residues in the reference polypeptide sequence, after aligning the sequences and introducing gaps, if necessary, to achieve the maximum percent sequence identity. To determine the sequence identity, the sequence of a query protein is aligned to the sequence of a reference protein or polypeptide. Methods for sequence alignment are well known in the art. For example, for determining the extent of an amino acid sequence identity of an arbitrary polypeptide relative to another amino acid sequence, the SIM Local similarity program as known in the art is preferably employed. For multiple alignment analysis, Clustal Omega is preferably used, as known to someone skilled in the art.

### DETAILED DESCRIPTION OF THE EMBODIMENTS OF THIS INVENTION

### Structural Characterization of Programmed Death Ligand 1 (PD-L1) Binding Proteins.

The protein as described herein comprises an amino acid sequence with at least 90% identity to the amino acid sequence of SEQ ID NO: 1 wherein the protein has (a) an aromatic amino acid selected from phenylalanine (F), tyrosine (Y), or tryptophane (W) at the position corresponding to position 6 of SEQ ID NO: 1, and (b) an acidic amino acid selected from aspartic acid (D) or glutamic acid (E), or a basic amino acid such as lysine (K) or arginine (R) at the position corresponding to position 8 of SEQ ID NO: 1.

In various embodiments, the protein is selected from amino acid sequences with at least 90%, 92%, 93%, 94%, 96%, 97%, or 98% identity to the amino acid sequence of SEQ ID NO: 1. The PD-L1 binding protein as described herein comprises an amino acid sequence with at least 90% identity to the amino acid sequence of SEQ ID NO: 1, wherein in preferred embodiments the protein has (a) a phenylalanine (F) at the position corresponding to position 6 of SEQ ID NO: 1, and (b) an aspartic acid (D) at the position corresponding to position 8 of SEQ ID NO: 1, and has a binding affinity for human PD-L1 of at least (or less than) 100 nM.

Specific examples for the protein as described herein that comprises an amino acid sequence with at least 90% identity to the amino acid sequence of SEQ ID NO: 1 are provided in SEQ ID NO: 2 and SEQ ID NO: 3.

As described herein, modifications in ubiquitin that result in high affinity binding to PD-L1 are located in the region comprising amino acid positions 6 and 8 of SEQ ID NO: 10, and may further comprise a modification in the region comprising amino acid position 62 of ubiquitin (SEQ ID NO: 10).

As a general concept, the present invention provides a protein comprising an amino acid sequence with at least 90% identity to the amino acid sequence of SEQ ID NO: 1, wherein the protein: (i) has an aromatic amino acid selected from phenylalanine (F), tyrosine (Y), or tryptophan (W) at the position corresponding to position 6 of SEQ ID NO: 1, and a charged amino acid selected from any one of aspartic acid (D), glutamic acid (E), lysine (K), arginine (R), and/or histidine (H) at the position corresponding to position 8 of SEQ ID NO: 1; and (ii) exhibits a binding affinity for PD-L1 of less than 100 nM.

### Aspects and Embodiments Concerning an Acidic Amino Acid at the Position Corresponding to Position 8 of SEQ ID NO: 1:

More specifically, the present invention provides a protein comprising an amino acid sequence with at least 90% identity to the amino acid sequence of SEQ ID NO: 1, wherein the protein: (i) has an aromatic amino acid selected from phenylalanine (F), tyrosine (Y), or tryptophan (W) at the position corresponding to position 6 of SEQ ID NO: 1, and an acidic amino acid selected from aspartic acid (D) or glutamic acid (E) at the position corresponding to position 8 of SEQ ID NO: 1; and (ii) exhibits a binding affinity for PD-L1 of less than 100 nM. In various embodiments, the said acidic amino acid is glutamic acid (E). In various preferred embodiments, the said acidic amino acid is aspartic acid (D).

In various other preferred embodiments, the said aromatic amino acid is phenylalanine (F), and the said acidic amino acid is aspartic acid (D) or glutamic acid (E). More preferably, the said aromatic amino acid is phenylalanine (F), and the said acidic amino acid is aspartic acid (D).

In various other preferred embodiments, the said aromatic amino acid is tyrosine (Y), and the said acidic amino acid is aspartic acid (D) or glutamic acid (E). More preferably, the said aromatic amino acid is tyrosine (Y), and the said acidic amino acid is aspartic acid (D).

In various other preferred embodiments, the said aromatic amino acid is tryptophan (W), and the said acidic amino acid is aspartic acid (D) or glutamic acid (E). More preferably, the said aromatic amino acid is tryptophan (W), and the said acidic amino acid is aspartic acid (D).

In various other embodiments, the protein comprising an amino acid sequence with at least 90% identity to the amino acid sequence of SEQ ID NO: 1 has an aromatic amino acid selected from phenylalanine (F), tyrosine (Y), or tryptophan (W) at the position corresponding to position 6 of SEQ ID NO: 1, an acidic amino acid selected from aspartic acid (D) or glutamic acid (E) at the position corresponding to position 8 of SEQ ID NO: 1, and an amino acid selected from tryptophan (W), glutamine (Q), or alanine (A) at the position corresponding to position 62 of SEQ ID NO: 1. In various embodiments, the said acidic amino acid is glutamic acid (E). In various preferred embodiments, the said acidic amino acid is aspartic acid (D).

In various other preferred embodiments, the said aromatic amino acid is phenylalanine (F), the said acidic amino acid is aspartic acid (D) or glutamic acid (E), and the amino acid at the position corresponding to position 62 of SEQ ID NO: 1 is tryptophan (W). More preferably, the said aromatic amino acid is phenylalanine (F), and the said acidic amino acid is aspartic acid (D), and the amino acid at the position corresponding to position 62 of SEQ ID NO: 1 is tryptophan (A).

In various other preferred embodiments, the said aromatic amino acid is phenylalanine (F), the said acidic amino acid is aspartic acid (D) or glutamic acid (E), and the amino acid at the position corresponding to position 62 of SEQ ID NO: 1 is glutamine (Q). More preferably, the said aromatic amino acid is phenylalanine (F), and the said acidic amino acid is aspartic acid (D), and the amino acid at the position corresponding to position 62 of SEQ ID NO: 1 is glutamine (Q).

In various other preferred embodiments, the said aromatic amino acid is phenylalanine (F), the said acidic amino acid is aspartic acid (D) or glutamic acid (E), and the amino acid at the position corresponding to position 62 of SEQ ID NO: 1 is alanine (A). More preferably, the said aromatic amino acid is phenylalanine (F), and the said acidic amino acid is aspartic acid (D), and the amino acid at the position corresponding to position 62 of SEQ ID NO: 1 is alanine (A). In the herein-described aspects and embodiments concerning SEQ ID NO: 1, including in particular the herein-described aspects and embodiments concerning an acidic amino acid at the position corresponding to position 8 of SEQ ID NO: 1, the protein may comprise an amino acid sequence with at least 92% identity to the amino acid sequence of SEQ ID NO: 1. Preferably, the protein may comprise an amino acid sequence with at least 93% identity to the amino acid sequence of SEQ ID NO: 1. More preferably, the protein may comprise an amino acid sequence with at least 94% identity to the amino acid sequence of SEQ ID NO: 1. Still more preferably, the protein may comprise an amino acid sequence with at least 96% identity to the amino acid sequence of SEQ ID NO: 1. Even more preferably, the protein may comprise an amino acid sequence with at least 97% or at least 98% identity to the amino acid sequence of SEQ ID NO: 1.

As further described herein, a protein comprising an amino acid sequence with at least 90% identity to the amino acid sequence of SEQ ID NO: 1 may in various embodiments describe a variant amino acid sequence (solely) based on amino acid substitutions as compared to the reference sequence(s) disclosed herein, in particular the sequence of SEQ ID NO: 1. Such proteins (or protein monomers) of the invention have a length of or comprise about 76 amino acids, as described elsewhere herein. In other embodiments, a protein comprising an amino acid sequence with at least 90% identity to the amino acid sequence of SEQ ID NO: 1 may in various embodiments describe a variant amino acid sequence (solely) based on amino acid substitutions as compared to the reference sequence(s) disclosed herein, in particular the sequence of SEQ ID NO: 1, as well as (N- or C-) terminal deletion(s) of any of 1, 2, 3, 4, or 5, amino acid residues. Such proteins (or protein monomers) of the invention have a length of or comprise about 71, 72, 73, 74, or 75 amino acids.

The herein-described aspects and embodiments concerning SEQ ID NO: 1, including in particular the herein-described aspects and embodiments concerning an acidic amino acid at the position corresponding to position 8 of SEQ ID NO: 1, also apply to multimeric forms disclosed herein comprising a protein comprising an amino acid sequence with at least 90% identity to the amino acid sequence of SEQ ID NO: 1 as described herein.

### Aspects and Embodiments Concerning a Basic Amino Acid at the Position Corresponding to Position 8 of SEQ ID NO: 1:

Further, the present invention also provides a protein comprising an amino acid sequence with at least 90% identity to the amino acid sequence of SEQ ID NO: 1, wherein the protein: (i) has an aromatic amino acid selected from phenylalanine (F), tyrosine (Y), or tryptophan (W) at the position corresponding to position 6 of SEQ ID NO: 1, and a basic amino acid selected from lysine (K), arginine (R), or histidine (H) at the position corresponding to position 8 of SEQ ID NO: 1; and (ii) exhibits a binding affinity for PD-L1 of less than 100 nM. In various embodiments, the said basic amino acid is histidine (H). In various preferred embodiments, the said basic amino acid is lysine (K) or arginine (R).

Accordingly, in various preferred embodiments, the said aromatic amino acid is phenylalanine (F), and the said basic amino acid is lysine (K) or arginine (R). More preferably, the said aromatic amino acid is phenylalanine (F), and the said basic amino acid is lysine (K).

In various other embodiments, the protein comprising an amino acid sequence with at least 90% identity to the amino acid sequence of SEQ ID NO: 1 has an aromatic amino acid selected from phenylalanine (F), tyrosine (Y), or tryptophan (W) at the position corresponding to position 6 of SEQ ID NO: 1, an a basic amino acid selected from lysine (K), arginine (R), or histidine (H) at the position corresponding to position 8 of SEQ ID NO: 1; and an amino acid selected from tryptophan (W), glutamine (Q), or alanine (A) at the position corresponding to position 62 of SEQ ID NO: 1. In various preferred embodiments, the said basic amino acid is lysine (K) or arginine (R).

Accordingly, in various preferred embodiments, the said aromatic amino acid is phenylalanine (F), the said basic amino acid is lysine (K) or arginine (R), and the amino acid at the position corresponding to position 62 of SEQ ID NO: 1 is tryptophan (W). More preferably, the said aromatic amino acid is phenylalanine (F), the said basic amino acid is lysine (K), and the amino acid at the position corresponding to position 62 of SEQ ID NO: 1 is tryptophan (W).

In all of the herein described aspects and embodiments concerning SEQ ID NO: 1, including in particular the herein-described aspects and embodiments concerning a basic amino acid at the position corresponding to position 8 of SEQ ID NO: 1, the protein may comprise an amino acid sequence with at least 92% identity to the amino acid sequence of SEQ ID NO: 1. Preferably, the protein may comprise an amino acid sequence with at least 93% identity to the amino acid sequence of SEQ ID NO: 1. More preferably, the protein may comprise an amino acid sequence with at least 94% identity to the amino acid sequence of SEQ ID NO: 1. Still more preferably, the protein may comprise an amino acid sequence with at least 96% identity to the amino acid sequence of SEQ ID NO: 1. Even more preferably, the protein may comprise an amino acid sequence with at least 97% or at least 98% identity to the amino acid sequence of SEQ ID NO: 1.

The herein-described aspects and embodiments concerning SEQ ID NO: 1, including in particular the herein-described aspects and embodiments concerning a basic amino acid at the position corresponding to position 8 of SEQ ID NO: 1, also apply to multimeric forms disclosed herein comprising a protein comprising an amino acid sequence with at least 90% identity to the amino acid sequence of SEQ ID NO: 1 as described herein.

The proteins of the present invention, including multimeric forms thereof disclosed herein, are considered to bind to the same or overlapping epitope of PD-L1. In various embodiments, the proteins of the present invention, including multimeric forms thereof disclosed herein, bind to or are considered to bind to the extracellular domain of PD-L1. In various embodiments, the proteins of the present invention including multimeric forms thereof disclosed herein, bind to or are considered to bind to the N-terminal region of the extracellular domain of PD-L1. More specifically, the PD-L1 binding proteins of the present invention including multimeric forms thereof disclosed herein, bind to or are considered to bind to an N-terminal region of the extracellular domain of PD-L1 comprising amino acid residues 1 to about 50 of SEQ ID NO: 11 (corresponding to amino acid residues 19 to about 68 of the amino acid sequence of PD-L1). The PD-L1 binding protein including multimeric forms thereof disclosed herein, may bind to an epitope in the N-terminal region of the extracellular domain of PD-L1 as described above, wherein the epitope comprises, or consists of, amino acid residues 9 to 15 of SEQ ID NO: 11 (corresponding to amino acid residues 27 to 33 of the amino acid sequence of PD-L1).

Functional characterization. The PD-L1 binding proteins as described herein, including multimeric forms thereof disclosed herein, have a binding affinity (KD) of less than (at least) 100 nM for PD-L1. In various embodiments, the protein binds PD-L1 with measurable binding affinity of any of less than 100 nM, less than 50 nM, less than 20 nM, less than 15 nM, less than 10 nM, less than 5 nM, less than 2 nM, and/or even less than 1 nM.

As described elsewhere herein, the lower the K_{D} value, the greater the binding affinity of the biomolecule for its binding partner. The higher the K_{D} value, the more weakly the binding partners bind to each other (see Examples). Accordingly, as described herein, the terms “less than 100 nM” and “at least 100 nM” may be used interchangeably herein since they both refer to the high binding affinity of the proteins of the invention for PD-L1. More specifically, in the context of high binding affinity, the terms “less than 100 nM” and “at least 100 nM” each mean a range of numerical values indicating K_{D} values <100 nM (=including 100 nM) because the lower the K_{D} value, the greater the binding affinity. The same considerations with regard to the interchangeable use of the terms “less than” and “at least” apply with regard to all preferred binding affinities for PD-L1 disclosed throughout the specification, e.g., “less than 15 nM” and “at least 15 nM” etc. Here again, the terms “less than 15 nM” and “at least 15 nM” each mean a range of numerical values indicating K_{D} values s 15 nM (=including 15 nM) because the lower the K_{D} value, the greater the binding affinity.

In preferred embodiments, the PD-L1 binding protein, including multimeric forms thereof disclosed herein, binds PD-L1 with measurable binding affinity of less than 15 nM. In various embodiments of the present invention, the binding affinity for PD-L1 of less than 100 nM as described above and throughout the specification means a binding affinity for human PD-L1 (hPD-L1) of less than (at least) 100 nM. Preferred binding affinities (less than 50 nM etc.) are described above and elsewhere herein and apply to embodiments in relation to binding of the proteins and multimers of the present invention to hPD-L1.

The appropriate methods are known to those skilled in the art or described in the literature. The methods for determining the binding affinities are known per se and can be selected for instance from the following methods known in the art: enzyme-linked immunosorbent assay (ELISA), surface plasmon resonance (SPR), kinetic exclusion analysis (KinExA assay), Bio-layer interferometry (BLI), flow cytometry, fluorescence spectroscopy techniques, isothermal titration calorimetry (ITC), analytical ultracentrifugation, radioimmunoassay (RIA or IRMA), and enhanced chemiluminescence (ECL). Some of the methods are described in the Examples below. Typically, the dissociation constant K_{D} is determined at 20° C., 25° C., or 30° C. If not specifically indicated otherwise, the K_{D} values recited herein are determined at 25° C. by SPR. The lower the K_{D} value, the greater the binding affinity of the biomolecule for its binding partner. The higher the K_{D} value, the more weakly the binding partners bind to each other (see Examples).

As disclosed herein, in the above-described aspects and embodiments, the protein may comprise an amino acid sequence with at least 92% identity or at least 93% identity or at least 94% identity to the amino acid sequence of SEQ ID NO: 1, and exhibits a binding affinity for PD-L1 of less than 100 nM. As further disclosed herein, in the above-described aspects and embodiments, the protein may comprise an amino acid sequence with at least 92% identity or at least 93% identity or at least 94% identity to the amino acid sequence of SEQ ID NO: 1, and preferably exhibits a binding affinity for PD-L1 of less than 50 nM. More preferably, the protein may comprise an amino acid sequence with at least 92% identity or at least 93% identity or at least 94% identity to the amino acid sequence of SEQ ID NO: 1, and exhibits a binding affinity for PD-L1 of less than 20 nM. Even more preferably, the protein may comprise an amino acid sequence with at least 92% identity or at least 93% identity or at least 94% identity to the amino acid sequence of SEQ ID NO: 1, and exhibits a binding affinity for PD-L1 of less than 15 nM or less than 10 nM. Still more preferably, in the above-described aspects and embodiments concerning acidic/basic amino acids at the position corresponding to position 8 of SEQ ID NO: 1, the protein may comprise an amino acid sequence with at least 92% identity or at least 93% identity or at least 94% identity to the amino acid sequence of SEQ ID NO: 1, and exhibits a binding affinity for PD-L1 of less than 5 nM. In particularly preferred embodiments, the protein may comprise an amino acid sequence with at least 92% identity or at least 93% identity or at least 94% identity to the amino acid sequence of SEQ ID NO: 1, and exhibits a binding affinity for PD-L1 of less than 2 nM, or even less than 1 nM.

As further disclosed herein, in the above-described aspects and embodiments, the protein may comprise an amino acid sequence with at least 96% identity or at least 97% identity or at least 98% identity to the amino acid sequence of SEQ ID NO: 1, and exhibits a binding affinity for PD-L1 of less than 100 nM. In the above-described aspects and embodiments, the protein may comprise an amino acid sequence with at least 96% identity or at least 97% identity or at least 98% identity to the amino acid sequence of SEQ ID NO: 1, and preferably exhibits a binding affinity for PD-L1 of less than 50 nM. More preferably, the protein may comprise an amino acid sequence with at least 96% identity or at least 97% identity or at least 98% identity to the amino acid sequence of SEQ ID NO: 1, and exhibits a binding affinity for PD-L1 of less than 20 nM. Even more preferably, the protein may comprise an amino acid sequence with at least 97% identity or at least 98% identity to the amino acid sequence of SEQ ID NO: 1, and exhibits a binding affinity for PD-L1 of less than 15 nM or less than 10 nM. Still more preferably, in the above-described aspects and embodiments, the protein may comprise an amino acid sequence with at least 97% identity or at least 98% identity to the amino acid sequence of SEQ ID NO: 1, and exhibits a binding affinity for PD-L1 of less than 5 nM. In particularly preferred embodiments, the protein may comprise an amino acid sequence with at least 97% identity or at least 98% identity to the amino acid sequence of SEQ ID NO: 1, and exhibits a binding affinity for PD-L1 of less than 2 nM, or even less than 1 nM.

As still further disclosed herein, in the aspects and embodiments concerning SEQ ID NO: 1 described above (see under “Structural characterization of PD-L1 binding proteins”), the protein, including multimeric forms thereof disclosed herein, preferably exhibits a binding affinity for PD-L1 of less than 50 nM. More preferably, the protein including multimeric forms thereof disclosed herein exhibits a binding affinity for PD-L1 of less than 20 nM. Even more preferably, the protein including multimeric forms thereof disclosed herein exhibits a binding affinity for PD-L1 of less than 15 nM or less than 10 nM. Still more preferably, the protein including multimeric forms thereof disclosed herein exhibits a binding affinity for PD-L1 of less than 5 nM. Particularly preferred is that the protein including multimeric forms thereof disclosed herein exhibits a binding affinity for PD-L1 of less than 2 nM, or even less than 1 nM.

The herein-described aspects and embodiments concerning functional characterization of the PD-L1 binding protein of the invention also apply to multimeric forms disclosed herein comprising a protein comprising an amino acid sequence with at least 90% identity to the amino acid sequence of SEQ ID NO: 1 as described herein.

Cellular PD-L1 binding of the protein as described herein can be determined by standard methods, including Immunofluorescence microscopy and flow cytometric analysis. In some embodiments, the specific PD-L1 binding of the proteins of the present invention is determined by cellular PD-L1 binding analysis. In the aspects and embodiments concerning SEQ ID NO: 1 described above (see under “Structural characterization of PD-L1 binding proteins”), the protein in various embodiments preferably exhibits a binding affinity for PD-L1 of less than 5 nM, preferably less than 3 nM, on PD-L1 expressing cells (see Examples). More specifically, in various embodiments the protein preferably exhibits a binding affinity for PD-L1 of less than 5 nM, preferably less than 3 nM, on PD-L1 expressing cells as determined by flow cytometry analysis (see Examples).

In some embodiments, the protein as described herein inhibits the interaction of PD-1 binding to PD-L1. In preferred embodiments, the proteins as described herein, including multimeric forms thereof disclosed herein, bind specifically to (human) PD-L1 with high affinity (i.e., less than 100 nM, preferably less than 50 nM, etc., as described elsewhere herein) and inhibit the interaction of PD-L1 with PD-1. As further described herein, inhibiting the interaction of PD-L1 with PD-1 in various embodiments means (or provides for) blocking the PD-1 inhibitory signalling.

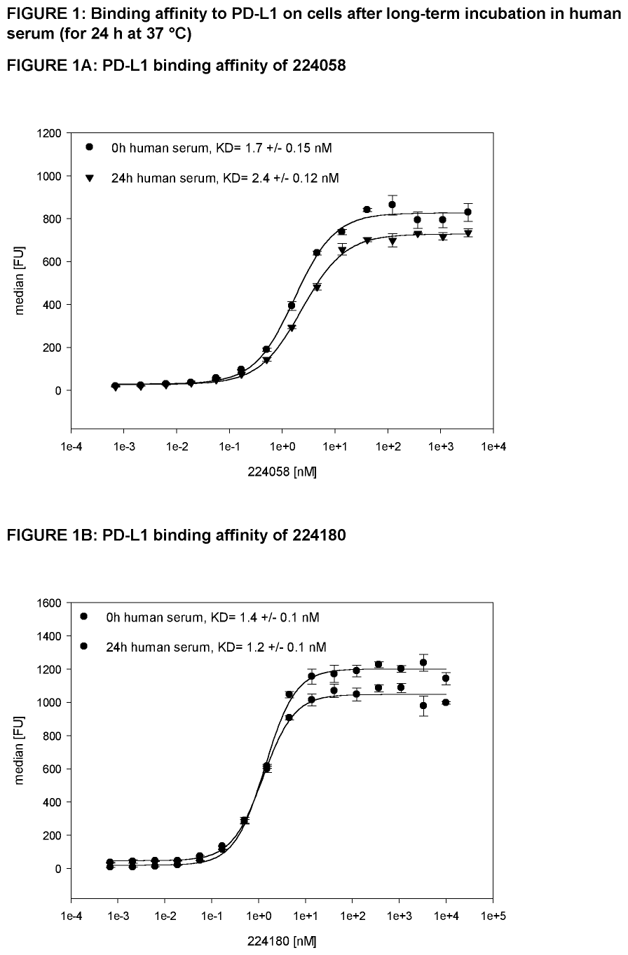

In some embodiments, the protein as described herein is particularly stable under different conditions. In preferred embodiments, the protein is stable in the presence of (human or mouse or rat) serum for at least 24 h at 37° C. In some embodiments, the protein as described herein is stable in the presence of human serum for at least 24 h at 37° C., as described in Example 6 and FIG. 1 in more detail. In some embodiments, the protein as described herein, is stable in the presence of mouse serum for at least 24 h at 37° C. For example, the stability of a PD-L1 binding protein can be determined by measuring the binding affinity (K_{D}) after incubation in serum for a long period of time at temperatures as high as 37° C. using standard methods as described herein above and in the Examples. In the aspects and embodiments concerning SEQ ID NO: 1 described above (see under “Structural characterization of PD-L1 binding proteins”), the protein in various embodiments preferably exhibits a binding affinity for PD-L1 of less than 5 nM, preferably less than 3 nM, on PD-L1 expressing cells, even after incubation in (human) serum for 24 h at 37° C. More specifically, in various embodiments the protein preferably exhibits a binding affinity for PD-L1 of less than 5 nM, preferably less than 3 nM, on PD-L1 expressing cells as determined by flow cytometry analysis, even after incubation in (human) serum for 24 h at 37° C.

In some embodiments, the PD-L1 binding protein as described herein is stable at high temperatures, preferably of at least 60° C. For stability analysis, for example spectroscopic or fluorescence-based methods in connection with chemical or physical unfolding are known to those skilled in the art. For example, the stability of a molecule can be determined by measuring the thermal melting (Tₘ) temperature, the temperature in ° Celsius (° C.) at which half of the molecules become unfolded, using standard methods. Typically, the higher the Tₘ, the more stable the molecule. Temperature stability was determined by differential scanning fluorimetry (DSF), as described in further detail in Example 3 and in Table 1.

Also the above-described further aspects and embodiments concerning functional characterization of the PD-L1 binding protein of the invention apply to multimeric forms disclosed herein comprising a protein comprising an amino acid sequence with at least 90% identity to the amino acid sequence of SEQ ID NO: 1 described herein throughout.

Multimers. The present invention provides multimers comprising a protein of the present invention as described above and throughout the present specification, i.e., as a general concept, the present invention provides a multimer comprising a protein comprising an amino acid sequence with at least 90% identity to the amino acid sequence of SEQ ID NO: 1, wherein the protein: (i) has an aromatic amino acid selected from phenylalanine (F), tyrosine (Y), or tryptophan (W) at the position corresponding to position 6 of SEQ ID NO: 1, and a charged amino acid selected from any one of aspartic acid (D), glutamic acid (E), lysine (K), arginine (R), and/or histidine (H) at the position corresponding to position 8 of SEQ ID NO: 1; and (ii) exhibits a binding affinity for Programmed (Cell) Death Ligand 1 (PD-L1) of less than 100 nM. More specifically, the present invention provides a multimer comprising a protein as described elsewhere herein in relation to “aspects and embodiments concerning an acidic amino acid at the position corresponding to position 8 of SEQ ID NO: 1”, and also provides a multimer comprising a protein as described elsewhere herein in relation to “aspects and embodiments concerning a basic amino acid at the position corresponding to position 8 of SEQ ID NO: 1”.

In the present invention, the multimer may be any of a dimer, a trimer, a tetramer, a pentamer, or a hexamer. In the present invention, the multimer preferably is a dimer or a trimer, more preferably a dimer. Accordingly, a multimer of the present invention comprises at least two domains or at least two monomers (=dimer). Accordingly, a protein of the present invention as described above throughout the present specification may be considered as a protein domain or protein monomer of a multimer provided by the present invention. Such protein domains or protein monomers have a length of or comprise about 76 amino acids.

In some embodiments, a multimer may comprise one, two, three, four, or more protein(s) as described herein. In one embodiment, the protein comprises 2, 3, 4, or more proteins linked to each other, i.e. the protein can be a dimer, trimer, or tetramer, etc. as described above, wherein at least one protein of the multimer is comprising an amino acid sequence of at least 90% identity to the amino acid sequence of SEQ ID NO: 1, wherein the protein has an aromatic amino acid selected from phenylalanine (F), tyrosine (Y), or tryptophane (W) at the position corresponding to position 6 of SEQ ID NO: 1, and an acidic amino acid selected from aspartic acid (D) or glutamic acid (E) at the position corresponding to position 8 of SEQ ID NO: 1 and wherein the multimeric protein has a binding affinity for human PD-L1 of less than 100 nM.

In one preferred embodiment, the PD-L1 binding protein comprises 2 proteins linked to each other wherein one protein of the multimer is comprising an amino acid sequence of at least 90% identity to the amino acid sequence of SEQ ID NO: 1, wherein the protein has an aromatic amino acid selected from phenylalanine (F), tyrosine (Y), or tryptophane (W) at the position corresponding to position 6 of SEQ ID NO: 1, and an acidic amino acid selected from aspartic acid (D) or glutamic acid (E) at the position corresponding to position 8 of SEQ ID NO: 1 and wherein the dimeric PD-L1 binding protein has a binding affinity for human PD-L1 of less than 100 nM. In one preferred embodiment, the PD-L1 binding protein comprises 2 proteins linked to each other wherein one protein has amino acid sequence of at least 90% identity to the amino acid sequence of SEQ ID NO: 1, wherein the protein has an aromatic amino acid selected from phenylalanine (F), tyrosine (Y), or tryptophane (W) at the position corresponding to position 6 of SEQ ID NO: 1, and an acidic amino acid selected from aspartic acid (D) or glutamic acid (E) at the position corresponding to position 8 of SEQ ID NO: 1, and the second protein has the amino acid sequence of at least 90% identity to SEQ ID NO: 4, and wherein the dimeric PD-L1 binding protein has a binding affinity for human PD-L1 of less than 100 nM. In various preferred embodiments of the present invention, the multimer comprises a protein of the present invention as described above and throughout the present specification, and a protein comprising an amino acid sequence with at least 90% identity to the amino acid sequence of SEQ ID NO: 4. In such case, the multimer of the present invention comprises at least two protein domains. The at least two protein domains of a multimer of the present invention are located N-terminally and C-terminally, respectively.

In one preferred embodiment, the PD-L1 binding protein comprises 2 proteins linked to each other wherein the protein comprising an amino acid sequence of at least 90% identity to the amino acid sequence of SEQ ID NO: 1, wherein the protein has an aromatic amino acid selected from phenylalanine (F), tyrosine (Y), or tryptophane (W) at the position corresponding to position 6 of SEQ ID NO: 1, and an acidic amino acid selected from aspartic acid (D) or glutamic acid (E) at the position corresponding to position 8 of SEQ ID NO: 1 is located N-terminal or C-terminal, preferably C-terminal, and wherein the dimeric PD-L1 binding protein has a binding affinity for human PD-L1 of less than 100 nM. In one preferred embodiment, the PD-L1 binding protein comprises 2 proteins linked to each other wherein the protein having amino acid sequence of at least 90% identity has an aromatic amino acid selected from phenylalanine (F), tyrosine (Y), or tryptophane (W) at the position corresponding to position 6 of SEQ ID NO: 1, and an acidic amino acid selected from aspartic acid (D) or glutamic acid (E) at the position corresponding to position 8 of SEQ ID NO: 1 is located N-terminal or C-terminal, preferably C-terminal, and wherein the second protein having the amino acid sequence of at least 90% identity to SEQ ID NO: 4 is located N-terminal or C-terminal, preferably N-terminal, and wherein the dimeric PD-L1 binding protein has a binding affinity for human PD-L1 of less than 100 nM.

In various embodiments, the multimer comprises a protein of the present invention as described above and throughout the present specification, and a protein comprising an amino acid sequence with at least 92% identity to the amino acid sequence of SEQ ID NO: 4. In various preferred embodiments, the multimer comprises a protein of the present invention as described above and throughout the present specification, and a protein comprising an amino acid sequence with at least 93% identity to the amino acid sequence of SEQ ID NO: 4. More preferably, the multimer comprises a protein of the present invention as described above and throughout the present specification, and a protein comprising an amino acid sequence with at least 94% identity to the amino acid sequence of SEQ ID NO: 4. Still more preferably, the multimer comprises a protein of the present invention as described above and throughout the present specification, and a protein comprising an amino acid sequence with at least 96% identity to the amino acid sequence of SEQ ID NO: 4. Even more preferably, the multimer comprises a protein of the present invention as described above and throughout the present specification, and a protein comprising an amino acid sequence with at least 97% identity to the amino acid sequence of SEQ ID NO: 4. In particularly preferred embodiments, the multimer comprises a protein of the present invention as described above and throughout the present specification, and a protein comprising an amino acid sequence with at least 98% or even 100% identity to the amino acid sequence of SEQ ID NO: 4.

Preferably, the multimer comprising a protein of the present invention as described above and throughout the present specification, and a protein comprising an amino acid sequence with at least 90% identity to the amino acid sequence of SEQ ID NO: 4 as described above, is a dimer or a trimer. More preferably, the multimer comprising a protein of the present invention as described above and throughout the present specification, and a protein comprising an amino acid sequence with at least 90% identity to the amino acid sequence of SEQ ID NO: 4 as described above, is a dimer.

Preferred embodiments relate to a dimeric PD-L1 binding protein comprising the amino acid sequence of SEQ ID NO: 4 and an amino acid sequence with at least 96% identity to the amino acid sequence of SEQ ID NO: 1, wherein the protein: (i) has an aromatic amino acid selected from phenylalanine (F), tyrosine (Y), or tryptophan (W) at the position corresponding to position 6 of SEQ ID NO: 1, and an acidic amino acid selected from aspartic acid (D) or glutamic acid (E) at the position corresponding to position 8 of SEQ ID NO: 1; and (ii) exhibits a binding affinity for human Programmed Death Ligand 1 (PD-L1) of less than 15 nM as determined by Surface Plasmon Resonance.

Preferably, in multimers of the present invention comprising a protein of the present invention as described above and throughout the present specification, and a protein comprising an amino acid sequence with at least 90% identity to the amino acid sequence of SEQ ID NO: 4 as described above, the protein (domain/monomer) of the present invention as described above and throughout the present specification is located C-terminally, and the protein (domain/monomer) comprising an amino acid sequence with at least 90% identity to the amino acid sequence of SEQ ID NO: 4 as described above is located N-terminally.

In one embodiment, the multimer is a dimer comprising of two proteins, wherein the second (C-terminal) protein corresponds to SEQ ID NO: 1 and the first (N-terminal) protein corresponds to SEQ ID NO: 4. The sequence of the dimer is SEQ ID NO: 5 (224039). In another embodiment, the multimer is a dimer comprising of two proteins, wherein the second (C-terminal) protein corresponds to SEQ ID NO: 2 and the first (N-terminal) protein corresponds to SEQ ID NO: 4. The sequence of the dimer is SEQ ID NO: 6 (224058). In another embodiment, the multimer is a dimer comprising of two proteins, wherein the second (C-terminal) protein corresponds to SEQ ID NO: 3 and the first (N-terminal) protein corresponds to SEQ ID NO: 4. The sequence of the dimer is SEQ ID NO: 7 (224180).

In some embodiments, the PD-L1 binding protein has at least 90% identity to SEQ ID NO: 5, SEQ ID NO: 6, or SEQ ID NO: 7. In preferred embodiments, the PD-L1 binding protein has at least 90% identity to SEQ ID NO: 5, SEQ ID NO: 6, or SEQ ID NO: 7, provided that the aromatic amino acid at the position corresponding to position 82 of SEQ ID NO: 5, SEQ ID NO: 6, or SEQ ID NO: 7 and the acidic amino acid at position 84 of of SEQ ID NO: 5, SEQ ID NO: 6, or SEQ ID NO: 7 remains unchanged.

In some embodiments, two or more proteins are directly linked. In some embodiments, two or more proteins as described herein are linked by a peptide linker. In various embodiments, two or more proteins as described herein are linked via a peptide linker of up to 30 amino acids. In other embodiments, two or more proteins as described herein are linked via a peptide linker of 10, 11, 12, 13, 14, 15, 16, 17, 18, 19, 20 amino acids.

A multimer as described above may be considered as a chimeric polypeptide comprising a first portion and a second portion, wherein said first portion comprises an amino acid sequence of a protein of the present invention as described above and throughout the present specification, and wherein said second portion comprises an amino acid sequence of a protein comprising an amino acid sequence with at least 90% identity to the amino acid sequence of SEQ ID NO: 4 as described above.

The functional characteristics of the protein of the present invention as described above and throughout the present specification fully apply to any of the multimers, fusion proteins and chimeric polypeptides provided by the present invention.

Coupling sites. In some embodiments, the PD-L1 binding protein as described herein further comprises one or more coupling site(s) for the coupling of chemical moieties. A coupling site is capable of reacting with other chemical groups to couple the PD-L1 binding protein to chemical moieties. The defined number and defined position of coupling sites enables site-specific coupling of chemical moieties to the PD-L1 binding protein as described herein. Thus, a large number of chemical moieties can be bound to the PD-L1 binding protein if required. The number of coupling sites can be adjusted to the optimal number for a certain application by a person skilled in the art to adjust the amount of the chemical moieties accordingly. In selected embodiments, the coupling site may be selected from the group of one or more amino acids which can be labeled with specific chemistry such as one or more cysteine residues, one or more lysine residues, one or more tyrosine, one or more tryptophan, or one or more histidine residues. The PD-L1 binding protein may comprise 1 to 20 coupling site(s), preferably 1 to 6 coupling site(s), preferably 2 coupling sites, or preferably one coupling site.

Coupling domains. One embodiment provides a PD-L1 binding protein that comprises at least one coupling domain of 1 to 80 amino acids comprising one or more coupling sites. In some embodiments, the coupling domain of 1 to 80 amino acids may comprise alanine, proline, or serine, and as coupling site cysteine. n other embodiments, the coupling domain of 5 to 80 amino acids may consist of alanine, proline, serine, and as coupling site cysteine. In one embodiment, the coupling domain is consisting of 20-60% alanine, 20-40% proline, 10-60% serine, and one or more cysteine as coupling site(s) at the C- or N-terminal end of the PD-L1 binding protein as described herein. In some embodiments the amino acids alanine, proline, and serine are randomly distributed throughout a coupling domain amino acid sequence so that not more than a maximum of 2, 3, 4, or 5 identical amino acid residues are adjacent, preferably a maximum of 3 amino acids. The composition of the 1 to 20 coupling domains can be different or identical.

Chemical moieties. In some embodiments, the chemical moieties are selected from any of chelators, drugs, toxins, dyes, and small molecules. In some embodiments, at least one of the chemical moieties is a chelator designed as a complexing agent for coupling one or more further moieties to the targeted compound to the PD-L1 binding protein as disclosed herein. One embodiment relates to the PD-L1 binding protein wherein the chelator is a complexing agent for coupling one or more radioisotopes or other detectable labels.

Diagnostic moiety. In some embodiments the protein comprising an amino acid sequence of at least 90% identity to the amino acid sequence of SEQ ID NO: 1, wherein the protein has an aromatic amino acid selected from phenylalanine (F), tyrosine (Y), or tryptophane (W) at the position corresponding to position 6 of SEQ ID NO: 1, and an acidic amino acid selected from aspartic acid (D) or glutamic acid (E) at the position corresponding to position 8 of SEQ ID NO: 1, further comprises at least one diagnostically active moiety. In various embodiments, the PD-L1 binding protein further comprises a diagnostic moiety. In other embodiments, the PD-L1 binding protein further comprises more than one diagnostic moiety. In some embodiments, such diagnostic moiety may be selected from radionuclides, fluorescent proteins, photosensitizers, dyes, fluorophore, enzymes, magnetic beads, metallic beads, colloidal particles, electron-dense reagent, biotin, digoxigenin, hapten, or any combination of the above. In some embodiments, a PD-L1 binding protein that comprises at least one diagnostic moiety can be employed, for example, as imaging agents, for example to evaluate presence of tumor cells or metastases, tumor distribution, and/or recurrence of tumor. Methods for detection or monitoring of cancer cells involve imaging methods. Such methods involve imaging PD-L1 related cancer cells by, for example, radioimaging or photoluminescense or fluorescence.

Therapeutic moiety. In some embodiments the protein comprising an amino acid sequence of at least 90% identity to the amino acid sequence of SEQ ID NO: 1, wherein the protein has an aromatic amino acid selected from phenylalanine (F), tyrosine (Y), or tryptophane (W) at the position corresponding to position 6 of SEQ ID NO: 1, and an acidic amino acid selected from aspartic acid (D) or glutamic acid (E) at the position corresponding to position 8 of SEQ ID NO: 1, further comprises at least one therapeutically active moiety. In other embodiments, the PD-L1 binding protein further comprises more than one therapeutically active moiety. In some embodiments, such therapeutically active moiety may be selected from a monoclonal antibody or a fragment thereof, an extracellular domain of a receptor or fragments thereof, a radionuclide, a cytotoxic compound, a cytokine, a chemokine, an enzyme, or derivatives thereof, or any combination of the above. In some embodiments, the PD-L1 binding protein that comprises a therapeutically active component may be used in targeted delivery of any of the above listed components to the PD-L1 expressing tumor cell and accumulate therein, thereby resulting in low levels of toxicity to normal cells.

Radionuclides. Suitable radionuclides for applications in imaging (for example, in vitro) or for radiotherapy include for example but are not limited to the group of gamma-emitting isotopes, the group of positron emitters, the group of beta-emitters, and the group of alpha-emitters. In some embodiments, suitable conjugation partners include chelators such as 1,4,7,10-tetraazacyclododecane-1,4,7,10-tetraacetic acid (DOTA) or diethylene triamine pentaacetic acid (DTPA) or their activated derivatives, nanoparticles and liposomes. In various embodiments, DOTA may be suitable as complexing agent for radioisotopes and other agents for imaging.

Moiety modulating pharmacokinetics. In some embodiments the protein comprising an amino acid sequence of at least 90% identity to the amino acid sequence of SEQ ID NO: 1, wherein the protein has an aromatic amino acid selected from phenylalanine (F), tyrosine (Y), or tryptophane (W) at the position corresponding to position 6 of SEQ ID NO: 1, and an acidic amino acid selected from aspartic acid (D) or glutamic acid (E) at the position corresponding to position 8 of SEQ ID NO: 1, further comprises at least one one moiety modulating pharmacokinetics. In some embodiments, the moiety modulating pharmacokinetic is selected from a polyethylene glycol, a human serum albumin, an albumin-binding peptide, an immunoglobulin binding peptide or an immunoglobulin or immunoglobulin fragments, a sialic acid, or a transferrin, a transferrin receptor binding protein, a polysaccharide (for example, hydroxylethyl starch), or an unstructured amino acid sequence which increases the hydrodynamic radius such as a multimer comprising amino acids alanine, glycine, serine, proline. In various embodiments, said moiety increases the half-life of the PD-L1 binding protein at least 1.5 fold. Several techniques for producing PD-L1 binding protein with extended half-life are known in the art, for example, direct fusions of the moiety modulating pharmacokinetics with the PD-L1 binding protein as described above or chemical coupling methods. The moiety modulating pharmacokinetics can be attached for example at one or several sites of the PD-L1 binding protein through a peptide linker sequence or through a coupling site as described above.

Conjugation of proteinaceous or non-proteinaceous moieties to the PD-L1 binding protein may be performed applying chemical methods well-known in the art. In some embodiments, coupling chemistry specific for derivatization of cysteine or lysine residues may be applicable. Chemical coupling can be performed by chemistry well known to someone skilled in the art, including but not limited to, substitution, addition or cycloaddition or oxidation chemistry (e.g. disulfide formation).

Molecules for purification/detection. In some embodiments, additional amino acids can extend either at the N-terminal end of the PD-L1 binding protein or the C-terminal end or both. Additional sequences may include for example sequences introduced e.g. for purification or detection. In one embodiment, additional amino acid sequences include one or more peptide sequences that confer an affinity to certain chromatography column materials. Typical examples for such sequences include, without being limiting, Strep-tags, oligohistidine-tags, glutathione S-transferase, maltose-binding protein, inteins, intein fragments, or the albumin-binding domain of protein G.

Compositions. Various embodiments relate to a composition comprising an amino acid sequence of at least 90% identity to the amino acid sequence of SEQ ID NO: 1, wherein the protein has an aromatic amino acid selected from phenylalanine (F), tyrosine (Y), or tryptophane (W) at the position corresponding to position 6 of SEQ ID NO: 1, and an acidic amino acid selected from aspartic acid (D) or glutamic acid (E) at the position corresponding to position 8 of SEQ ID NO: 1 and wherein the protein has a binding affinity for human PD-L1 of less than 100 nM. Various embodiments relate to a composition comprising the PD-L1 binding protein as defined above for use in medicine. Various preferred embodiments relate to a composition comprising the PD-L1 binding protein as defined above for use in the diagnosis or treatment of various PD-L1 related (PD-L1 positive) tumors or infectious diseases such a chronic viral infection. Compositions comprising the PD-L1 binding protein as described above may be used for clinical applications for both diagnostic and therapeutic purposes. In particular, compositions comprising the PD-L1 binding protein as described above may be used for clinical applications for imaging, monitoring, and eliminating or inactivating pathological cells that express PD-L1.

Various embodiments relate to a diagnostic composition for the diagnosis of PD-L1 related cancer comprising the PD-L1 binding protein as defined herein and a diagnostically acceptable carrier and/or diluent. These include for example but are not limited to stabilizing agents, surface-active agents, salts, buffers, coloring agents etc. The compositions can be in the form of a liquid preparation, a lyophilisate, granules, in the form of an emulsion or a liposomal preparation.

The diagnostic composition comprising the PD-L1 binding protein as described herein can be used for diagnosis of PD-L1 related cancer, as described above.

Various embodiments relate to a pharmaceutical (e.g. therapeutical) composition for the treatment of diseases comprising the PD-L1 binding protein as disclosed herein, and a pharmaceutically (e.g. therapeutically) acceptable carrier and/or diluent. The pharmaceutical (e.g. therapeutical) composition optionally may contain further auxiliary agents and excipients known per se. These include for example but are not limited to stabilizing agents, surface-active agents, salts, buffers, coloring agents etc.

The pharmaceutical composition comprising the PD-L1 binding protein as defined herein can be used for treatment of diseases, as described above.

The compositions contain an effective dose of the PD-L1 binding protein as defined herein. The amount of protein to be administered depends on the organism, the type of disease, the age and weight of the patient and further factors known per se. Depending on the galenic preparation these compositions can be administered parenterally by injection or infusion, systemically, intraperitoneally, intramuscularly, subcutaneously, transdermally, or by other conventionally employed methods of application.

The composition can be in the form of a liquid preparation, a lyophilisate, a cream, a lotion for topical application, an aerosol, in the form of powders, granules, in the form of an emulsion or a liposomal preparation. The type of preparation depends on the type of disease, the route of administration, the severity of the disease, the patient and other factors known to those skilled in the art of medicine.

The various components of the composition may be packaged as a kit with instructions for use. Use in medicine. Various embodiments relate to the PD-L1 binding protein as disclosed herein for use in medicine. In one embodiment, the PD-L1 binding protein is used in medicine to diagnose or treat cancer associated with PD-L1 expression. Accordingly, disclosed herein is a method of diagnosis or treatment of PD-L1 related cancer or infectious diseases. The PD-L1 binding proteins as disclosed herein allow selective diagnosis and treatment of PD-L1 related cancer cells or cancer tissues, for example, from melanoma, NSCLC, head and neck cancer, bladder cancer, breast cancer, and others. PD-L1 is known to be upregulated in tumor cells, possibly resulting in uncontrolled growth of tumor cells and in the formation of metastases. The PD-L1 binding proteins as disclosed herein further allow selective diagnosis and treatment of PD-L1 related infectious diseases such as chronic viral infection (HIV, HBV, HCV). In one embodiment, the PD-L1 binding protein is used to diagnose PD-L1 related cancer or infectious diseases by applying in vitro methods.

One embodiment is a method of diagnosing (including monitoring) a subject having PD-L1 related cancer or an infectious disease such as chronic viral infection, the method of diagnosis (monitoring) comprising administering to the subject the PD-L1 binding protein as described herein, optionally conjugated to radioactive molecules. In various embodiments, the PD-L1 binding protein as disclosed herein may be used for diagnosis of PD-L1 related cancer or infectious diseases such as chronic viral infection, optionally wherein the PD-L1 binding protein is conjugated to a radioactive molecule. In some embodiments, the PD-L1 binding protein is used in (in vitro) imaging methods with labels such as radioactive or fluorescent and can be employed to visualize PD-L1 on specific tissues or cells, for example, to evaluate presence of PD-L1 related tumor cells (PD-L1 positive tumor cells), PD-L1 related tumor distribution, recurrence of PD-L1 related tumor (PD-L1 positive tumors), and/or to evaluate the response of a patient to a therapeutic treatment.

One embodiment is a method of treating a subject having PD-L1 related (positive) cancer (tumor) or an infectious disease such as chronic viral infection, the method of treatment comprising administering to the subject the PD-L1 specific binding protein as described herein, optionally conjugated to a radioactive molecule and/or a cytotoxic agent, or as immunooncological agent. In various embodiments, the PD-L1 binding protein as disclosed herein may be used for treatment of PD-L1 related cancer or infectious diseases such as chronic viral infection, optionally wherein the PD-L1 binding protein is conjugated to a cytotoxic agent and/or to a radioactive molecule or expressed on the surface of target specific CarT cells. Some embodiments relate to the use of the PD-L1 binding protein labelled with a suitable radioisotope or cytotoxic compound or treatment of PD-L1 related (positive) tumor cells, in particular to control or kill PD-L1 related (positive) tumor cells, for example malignant cells. In one embodiment, curative doses of radiation are selectively delivered of to PD-L1 related (positive) tumor cells but not to normal cells.

As further described herein, in various embodiments, a PD-L1 related cancer is characterized by cancer cells expressing, or overexpressing, PD-L1. In various embodiments, the PD-L1 related cancer is a solid tumor expressing, or overexpressing, PD-L1. A use as immunooncology agent refers to a use of the PD-L1 binding protein as disclosed herein as therapeutical composition for the treatment of diseases, i.e. to promote an immune response against cancer. Certain cancer cells act to inhibit an immune response against that cell, such as by inhibiting a T cell response against the cell. The PD-L1 binding protein as disclosed herein as part of a medical composition for use as therapeutic agent may counteract the inhibition of the immune response.

Producing PD-L1 binding proteins. PD-L1 binding proteins as described herein may be prepared by any of the many conventional and well known techniques such as plain organic synthetic strategies, solid phase-assisted synthesis techniques, fragment ligation techniques or by commercially available automated synthesizers. On the other hand, they may also be prepared by conventional recombinant techniques alone or in combination with conventional synthetic techniques. Furthermore, they may also be prepared by cell-free in vitro transcription/translation.

Various embodiments relate to a polynucleotide encoding a PD-L1 binding protein as disclosed herein. One embodiment further provides an expression vector comprising said polynucleotide, and a host cell comprising said isolated polynucleotide or the expression vector.

Various embodiments relate to a method of producing a PD-L1 binding protein as disclosed herein comprising the steps of a) culturing of a host cell under suitable conditions which allow expression of said protein and b) isolating said protein.

For example, one or more polynucleotides which encode for the PD-L1 binding protein may be expressed in a suitable host and the produced PD-L1 binding protein can be isolated. A host cell comprises said nucleic acid molecule or vector. Suitable host cells include prokaryotes or eukaryotes. A vector means any molecule or entity (e.g., nucleic acid, plasmid, bacteriophage or virus) that can be used to transfer protein coding information into a host cell. Various cell culture systems, for example but not limited to mammalian, yeast, plant, or insect, can also be employed to express recombinant proteins. Suitable conditions for culturing prokaryotic or eukaryotic host cells are well known to the person skilled in the art. Cultivation of cells and protein expression for the purpose of protein production can be performed at any scale, starting from small volume shaker flasks to large fermenters, applying technologies well-known to any skilled in the art.

One embodiment is directed to a method of producing a protein as detailed above, said method comprising the following steps: (a) preparing a nucleic acid encoding a PD-L1 binding protein as defined herein; (b) introducing said nucleic acid into an expression vector; (c) introducing said expression vector into a host cell; (d) cultivating the host cell; (e) subjecting the host cell to culturing conditions under which a PD-L1 binding protein is expressed, thereby producing a PD-L1 binding protein as defined herein; (f) optionally isolating the PD-L1 binding protein produced in step (e); and (g) optionally conjugating the PD-L1 binding protein with further functional moieties as defined herein.

In general, isolation of purified PD-L1 binding protein from the cultivation mixture can be performed applying conventional methods and technologies well known in the art, such as centrifugation, precipitation, flocculation, different embodiments of chromatography, filtration, dialysis, concentration and combinations thereof, and others. Chromatographic methods are well-known in the art and comprise without limitation ion exchange chromatography, gel filtration chromatography (size exclusion chromatography), hydrophobic interaction chromatography or affinity chromatography.

For simplified purification, the PD-L1 binding protein can be fused to other peptide sequences having an increased affinity to separation materials. Preferably, such fusions are selected that do not have a detrimental effect on the functionality of the PD-L1 binding protein or can be separated after the purification due to the introduction of specific protease cleavage sites. Such methods are also known to those skilled in the art.

Methods to detect PD-L1 in a sample. Some embodiments relate to detect PD-L1 in a sample, the method comprising detecting the binding of PD-L1 with a PD-L1 binding protein as described above in the sample by contacting the sample with a PD-L1 binding protein as described above. Some embodiments, the protein as described herein is used in methods to determine the presence of PD-L1. Some embodiments relate to a method of analyzing the presence of PD-L1 in a sample, the method comprising the following steps: (i) providing a sample that contains PD-L1, (ii) providing the binding protein for PD-L1, (iii) contacting the sample that contains PD-L1 with the binding protein for PD-L1 as described herein under conditions that permit binding of the at least one protein to PD-L1, (iv) isolating (eluting) the complex of a PD-L1 and the binding protein for PD-L1, and (v) determining the amount of the binding protein for PD-L1 which indicates the amount of PD-L1 in the sample of (i). In some embodiments, the sample may be a liquid sample, such as a blood sample or a urine sample or a tumor sample in liquid.

### EXAMPLES

The following Examples are provided for further illustration of the invention. The invention, however, is not limited thereto, and the following Examples merely show the practicability of the invention on the basis of the above description.

### Example 1. Expression and Purification of PD-L1 Binding Proteins

The genes for proteins 224039, 224058, 224180, and 224121 (SEQ ID NOs: 5-8) were cloned into an expression vector using standard methods known to a skilled person, purified and analyzed as described below. All proteins were expressed and highly purified by affinity chromatography and gel filtration. After affinity chromatography purification a size exclusion chromatography (SEC) was performed using an Akta system and a Superdex™ 200 HiLoad 16/600 column (GE Healthcare). The column had a volume of 120 ml and was equilibrated with 2 CV. The samples were applied with a flow rate of 1 ml/min using PBS as running buffer. Fraction collection started as the signal intensity reached 10 mAU. Following SDS-PAGE analysis positive fractions were pooled and their protein concentrations were measured.

Purity was confirmed by SDS-PAGE, SE-HPLC and RP-HPLC. The purity was at least 98% as determined by RP-HPLC. Protein concentrations were determined by absorbance measurement at 280 nm using the specific molar absorbent coefficient. RP chromatography (RP HPLC) was performed using an Ultimate 3000 HPLC system (Thermo Fisher Scientific) and a PLRP-S (5 μm, 300 Å) column (Agilent).

### Example 2: Mammalian Expression of Human and Mouse PD-L1

Expi293-F-cells were cultured with 0.5-1 Mio cells/ml in Expi293-F expression medium (Fisher scientific, 13489756) in shake flasks at 135 rpm, 37° C., 8% CO₂ and 95% humidity. 1 day before transfection, cells were seeded with a density of 2.0 Mio cells/ml. On the day of transfection, cells were seeded with a density of 2.5 Mio cells/ml. 1 μg plasmid-DNA of extracellular domain of hPD-L1-Fc or mPD-L1-Fc per ml of culture volume were diluted in Opti-MEM I Reduced Serum Medium (Life Technologies, 31985-062). ExpiFectamine was diluted in Opti-MEM I Reduced Serum Medium, according to manufacturer information and incubated for 5 min at rt. Subsequently, DNA-solution was added to the ExpiFectamine-mixture, incubated for 20 min at rt and mixed with the cells. After 16 h enhancer was added to the cells. Supernatant was collected after 96-120 h, centrifuged and filtered through a 0.45 μm membrane.

### Example 3. Stability at High Temperatures (Above 60° C.)

Thermal stability of the proteins was determined by Differential Scanning Fluorimetry (DSF). Each probe was transferred at concentrations of 0.1 μg/μL to a MicroAmp Optical 96-well plate, and SYPRO Orange dye was added at suitable dilution. A temperature ramp from 25 to 95° C. was programmed with a heating rate of 1° C./min. Fluorescence was constantly measured at an excitation wavelength of 520 nm and the emission wavelength at 623 nm (ViiA 7 Real-Time PCR System (Thermo Scientific). The midpoints of transition for the thermal unfolding (Tm, melting points) were determined and are shown in Table 1.

### Example 4. Biochemical Binding Analysis (Surface Plasmon Resonance, SPR)

Recombinant protein A was immobilized on a High Capacity Amine sensor chip (Bruker) after NHS/EDC activation resulting in approx. 1000 RU with a Sierra SPR-32 system (Bruker). The chip was equilibrated with SPR running buffer (PBS 0.05%, Tween pH 7.3). Injection of ethanolamine after ligand immobilization was used to block unreacted NHS groups. The Fc-tagged extracellular domain of hPD-L1 and mPD-L1 were injected with 30 nM followed by the injection of PD-L1 binding proteins with a flow rate of 30 μl/min. A flow cell without ligand was used as reference. Upon ligand binding, protein analyte was accumulated on the surface increasing the refractive index. This change in the refractive index was measured in real time and plotted as response or resonance units (RU) versus time. The analytes were applied to the chip in serial dilutions. The association was performed for 120 seconds and the dissociation for 180 seconds. After each run, the chip surface was regenerated with 30 μl regeneration buffer (10 mM glycine pH 2.0) and equilibrated with running buffer. Binding studies were carried out by the use of the Sierra SPR-32 system (Bruker) and data evaluation was operated via the Sierra Analyser software, provided by the manufacturer.

Table 1 shows the binding affinity to PD-L1. The binding to mouse PD-L1 could be detected for 211828 but no valid KD could be determined with SPR.

TABLE 1
Binding affinity (K_{D}) and temperature stability.

| | | | SPR | SPR | DSF |
|---|---|---|---|---|---|
| SEQ | comprising | | KD vs | KD vs | Thermal |
| ID NO: | SEQ ID NOs: | CID | hPD-L1 | mPD-L1 | transition |
| 5 | 4 and 1 | 224039 | 0.9 nM | 91 nM | 68° C. |
| 6 | 4 and 2 | 224058 | 0.9 nM | 88 nM | 62° C. |
| 7 | 4 and 3 | 224180 | 11 nM | 91 nM | 63° C. |
| 8 | 4 and 13 | 224121 | 49 nM | 102 nM | 72° C. |
| 9 | 4 and 12 | 211828 | 84 nM | detected | 71° C. |

### Example 5. Functional Characterization: Specific Binding to Cell Surface Expressed hPD-L1 and mPD-L1 (Flow Cytometry)

Flow cytometry was used to analyze the specific interaction of PD-L1 binding proteins with surface-exposed human PD-L1 (hPD-L1) or mouse PD-L1 (mPD-L1). Transfected HEK293-hPD-L1-cells, HEK293-mPD-L1-cells, empty vector control HEK293-pEntry-cells and the native hPD-L1-expressing H460-cell line were trypsinized and resuspended in medium containing FCS and washed in pre-cooled FACS blocking buffer. A cell concentration of 1 Mio cells/ml was prepared for cell staining and filled with 100 μl/well into a 96 well plate (Greiner) in triplicate for each cell line. A dilution series of proteins or 1 μg/ml Avelumab (Merck) as positive control was added to PD-L1 expressing cells and control cells. After 45 min the supernatants were removed, and 100 μl/well rabbit anti-Strep-Tag antibody (GenScript; A00626), 1:300 diluted in FACS blocking buffer, were added to the wells with diluted binding proteins. Avelumab was detected with anti-human-IgG-Alexa 488 (Invitrogen; A-11013) with a dilution of 1:1000. After removal of the anti-Strep-Tag antibody, goat anti-rabbit IgG Alexa Fluor 488 antibody (Invitrogen; A11008) was applied in a 1:1000 dilution to the other wells. Flow cytometry measurement was conducted on the Guava easyCyte 5HT device (Merck-Millipore) at excitation wavelength 488 nm and emission wavelength 520 nm.

224039, 224058, 224180 (SEQ ID NOs: 5-7) showed specific binding on human PD-L1 or mouse PD-L1 overexpressing HEK293-cells. No binding could be detected on HEK293-pEntry-cells. 224039, 224058, 224180 show also a significant binding to native human PD-L1-expressing cells whereas 224121 and 211828 show only weak binding. In Table 2, Pos 6 and Pos 8 refer to the positions in the second (C-terminal) monomer of the protein.

TABLE 2
Cell binding analysis (Flow cytometry)

| Clone | SEQ | Pos | Pos | HEK293- | HEK293- | HEK293- | |
|---|---|---|---|---|---|---|---|
| ID | ID | 6 | 8 | hPD-L1 | mPD-L1 | pEntry | H460 |
| 224039 | 5 | F | D | +++ | ++ | − | + |
| 224058 | 6 | F | D | +++ | ++ | − | + |
| 224180 | 7 | F | D | +++ | ++ | − | + |
| 224121 | 8 | H | A | ++ | + | − | (+) |
| 211828 | 9 | R | T | + | + | − | (+) |
| ubiqui- | 10 | T | T | − | − | − | − |
| tin | | | | | | | |

### Example 6. Binding Affinity to PD-L1 in Human Serum after Long-Term Incubation (Cell Binding Assay—Flow Cytometry)

Dilution series from 3.3×10⁻⁶ M to 7×10⁻¹³ M of 224058 (SEQ ID NO: 6) and dilution series from 1×10⁻⁵ M to 7×10⁻¹³ M of 224180 (SEQ ID NO: 7) or of 224039 (SEQ ID NO: 5) were incubated in human serum for 0 h and 24 h at 37° C. PD-L1 overexpressing HEK293-cells were trypsinized, washed with FACS blocking buffer, seeded in 96-well round bottom plates with a density of 0.1 Mio cells/100 μl and protein dilution series were added to the cells. After 45 min the supernatants were removed, and cells were washed. Binding was proven with 100 μl/well rabbit anti-Strep-Tag antibody 1:300 diluted in FACS blocking buffer in a first step and goat anti-rabbit IgG Alexa Fluor 488 antibody in a 1:1000 dilution in a second step. The read out was described above. The proteins are stable in human serum for at least 24 h (FIG. 1) with only minor loss of binding affinity to PD-L1 expressed on cells (224058: K_{D} for human PD-1 after 24 h 2.4 nM; 224180: K_{D} for human PD-1 after 24 h 1.2 nM). 224039 is stable in human serum even after 24 h incubation with K_{D} for human PD-L1 of 1.3 nM.

### Example 7. Serum Stability of PD-L1 Binding Proteins (ELISA)

High binding plates (Greiner, 781061) were immobilized with 2.5 μg/ml recombinant human PD-L1-Fc over night at 4° C. Dilution series of 1 μM to 7×10⁻⁸ μM of 224058 (SEQ ID NO: 6), 224180 (SEQ ID NO: 7) and 224039 (SEQ ID NO: 5) were incubated in 100% mouse serum or 100% rat serum for 24 h at 37° C. ELISA-plates were washed 3 times with PBST (PBS, 0.1% Tween) and blocked with 3% BSA/0.5% Tween/PBS 2 h at RT. Dilution series after 0 h or 24 h incubation in the presence of serum were incubated on ELISA-plates 1 h at rt. After washing with PBST, wells were incubated with biotinylated anti-ubiquitin-antibody (1:300) for 1 h at rt. The binding was visualized with Streptavidin-HRP (1:5.000). The PD-L1 binding proteins are stable in rat and mouse serum. They show no significant change in K_{D} after 24 h serum incubation (Table 3).

TABLE 3
Stability of binding proteins in serum.

| | | serum stability in rat serum | | | serum stability in mouse serum | | |
|---|---|---|---|---|---|---|---|
| | | Kd 0 h | Kd 24 h | increasing | Kd 0 h | Kd 24 h | increasing |
| SEQ | CID | [nM] | [nM] | of KD [fold] | [nM] | [nM] | of KD [fold] |
| 7 | 224180 | 0.21 | 0.26 | 1.25 | 0.14 | 0.08 | 0.60 |
| 6 | 224058 | 0.29 | 0.37 | 1.28 | 0.23 | 0.17 | 0.74 |
| 5 | 224039 | 0.27 | 0.25 | 0.91 | 0.18 | 0.17 | 0.90 |

Appendix - SEQUENCES
(224039-2)
SEQ ID NO: 1
MQIFVFTDTGKTITLEVEPSDTIENVKAKIQDKEGIPPDQQRLIW
AGKQLEDGRTLSDYNIWPRRLLHLVLRLRAA
(224058-2)
SEQ ID NO: 2
MQIFVFTDTGKTITLEVEPSDTIENVKAKIQDKEGIPPDQQRLIW
AGKQLEDGRTLSDYNIQPRRLLHLVLRLRAA
(224180-2)
SEQ ID NO: 3
MQIFVFTDTGKTITLEVEPSDTIENVKAKIQDKEGIPPDQQRLIW
AGKQLEDGRTLSDYNIAPRRLLHLVLRLRAA
(224039-1/224058-1/224180-1/224121-1/211828-1)
SEQ ID NO: 4
MQIFVDTLTGKTITLEVEPSDTIENVKAKIQDKEGIPPDQQRLIW
AGKQLEDGRTLSDYNIRYPAFLHLVLRLRAA
(224039)
SEQ ID NO: 5
MQIFVDTLTGKTITLEVEPSDTIENVKAKIQDKEGIPPDQQRLIW
AGKQLEDGRTLSDYNIRYPAFLHLVLRLRAAMQIFVFTDTGKTIT
LEVEPSDTIENVKAKIQDKEGIPPDQQRLIWAGKQLEDGRTLSDY
NIWPRRLLHLVLRLRAA
(224058)
SEQ ID NO: 6
MQIFVDTLTGKTITLEVEPSDTIENVKAKIQDKEGIPPDQQRLIW
AGKQLEDGRTLSDYNIRYPAFLHLVLRLRAAMQIFVFTDTGKTIT
LEVEPSDTIENVKAKIQDKEGIPPDQQRLIWAGKQLEDGRTLSDY
NIQPRRLLHLVLRLRAA
(224180)
SEQ ID NO: 7
MQIFVDTLTGKTITLEVEPSDTIENVKAKIQDKEGIPPDQQRLIW
AGKQLEDGRTLSDYNIRYPAFLHLVLRLRAAMQIFVFTDTGKTIT
LEVEPSDTIENVKAKIQDKEGIPPDQQRLIWAGKQLEDGRTLSDY
NIAPRRLLHLVLRLRAASAWSHPQFEK
(224121)
SEQ ID NO: 8
MQIFVDTLTGKTITLEVEPSDTIENVKAKIQDKEGIPPDQQRLIW
AGKQLEDGRTLSDYNIRYPAFLHLVLRLRAAMQIFVHTATGKTIT
LEVEPSDTIENVKAKIQDKEGIPPDQQRLIWAGKQLEDGRTLSDY
NIWPRRLLHLVLRLRAA
(211828)
SEQ ID NO: 9
MQIFVDTLTGKTITLEVEPSDTIENVKAKIQDKEGIPPDQQRLIW
AGKQLEDGRTLSDYNIRYPAFLHLVLRLRAAMQIFVRTTTGKTIT
LEVEPSDTIENVKAKIQDKEGIPPDQQRLIWAGKQLEDGRTLSDY
NIWPRRLLHLVLRLRAA
(ubiquitin)
SEQ ID NO: 10
MQIFVKTLTGKTITLEVEPSDTIENVKAKIQDKEGIPPDQQRLIF
AGKQLEDGRTLSDYNIQKESTLHLVLRLRAA
(extracellular domain of hPD-L1)
SEQ ID NO: 11
FTVTVPKDLYVVEYGSNMTIECKFPVEKQLDLAALIVYWEMEDKN
IIQFVHGEEDLKVQHSSYRQRARLLKDQLSLGNAALQITDVKLQD
AGVYRCMISYGGADYKRITVKVNAPYNKINQRILVVDPVTSEHEL
TCQAEGYPKAEVIWTSSDHQVLSGKTTTTNSKREEKLFNVTSTLR
INTTTNEIFYCTFRRLDPEENHTAELVIPELPLAHPPNER
(224121-2)
SEQ ID NO: 12
MQIFVHTATGKTITLEVEPSDTIENVKAKIQDKEGIPPDQQRLIW
AGKQLEDGRTLSDYNIWPRRLLHLVLRLRAA
(211828-2)
SEQ ID NO: 13
MQIFVRTTTGKTITLEVEPSDTIENVKAKIQDKEGIPPDQQRLIW
AGKQLEDGRTLSDYNIWPRRLLHLVLRLRAA

1. A dimeric PD-L1 binding protein comprising the amino acid sequence of SEQ ID NO: 4 and an amino acid sequence with at least 96% identity to the amino acid sequence of SEQ ID NO: 1, wherein the protein:
(i) has an aromatic amino acid selected from phenylalanine (F), tyrosine (Y), or tryptophan (W) at the position corresponding to position 6 of SEQ ID NO: 1, and an acidic amino acid selected from aspartic acid (D) or glutamic acid (E) at the position corresponding to position 8 of SEQ ID NO: 1; and
(ii) exhibits a binding affinity for human Programmed Death Ligand 1 (PD-L1) of less than 15 nM as determined by Surface Plasmon Resonance.

2. The dimeric PD-L1 binding protein according to claim 1, wherein the protein has a phenylalanine (F) at the position corresponding to position 6 of SEQ ID NO: 1, and an aspartic acid (D) at the position corresponding to position 8 of SEQ ID NO: 1.

3. The dimeric PD-L1 binding protein according to claim 1, wherein the dimeric PD-L1 binding protein is stable in serum after 24 h incubation at 37° C.

4. The dimeric PD-L1 binding protein according to claim 1, further comprising one or more coupling sites for the coupling of chemical moieties.

5. The dimeric PD-L1 binding protein according to claim 1, further comprising at least one diagnostically active moiety or at least one therapeutically active moiety.

6. The dimeric PD-L1 binding protein according to claim 1, further comprising at least one moiety modulating pharmacokinetics.

7-10. (canceled)

11. A method for producing the dimeric PD-L1 binding protein according to claim 1, comprising: a) culturing a host cell that produces the dimeric PD-L1 binding protein according to claim 1 under conditions suitable to obtain said dimeric PD-L1 binding protein; and b) isolating said dimeric PD-L1 binding protein that is produced.

12. A method of detecting PD-L1 in a sample, the method comprising contacting the sample with the dimeric PD-L1 binding protein according to claim 1 under conditions sufficient to form a complex between PD-L1 and the dimeric PD-L1 binding protein according to claim 1 and detecting the complex, whereby PD-L1 in the sample is detected.

13. The dimeric PD-L1 binding protein according to claim 4, wherein the chemical moieties are selected from the group consisting of chelators, drugs, toxins, dyes, and small molecules.

14. The dimeric PD-L1 binding protein according to claim 6, wherein the at least one moiety modulating pharmacokinetics is selected from the group consisting of a serum albumin; an albumin binding protein; an immunoglobulin binding protein; an immunoglobulin or immunoglobulin fragment; a polysaccharide; an unstructured amino acid sequence comprising amino acids alanine, glycine, serine, and proline; a polyethylene glycol; a sialic acid; a transferrin; and a transferrin receptor binding protein; or any combination thereof.

15. A method for detecting a PD-L1-positive tumor, the method comprising contacting a sample suspected of containing a PD-L1-positive tumor with the dimeric PD-L1 binding protein according to claim 1 and detecting a complex formed between the PD-L1-positive tumor and the dimeric PD-L1 binding protein according to claim 1, wherein the dimeric PD-L1 binding protein according to claim 1 comprises a detectable moiety.

16. The method according to claim 15, wherein the detectable moiety is selected from the group consisting of a radionuclide, a fluorescent protein, a photosensitizer, a dye, a fluorophore, an enzyme, a magnetic bead, a metallic bead, a colloidal particle, an electron-dense reagent, biotin, digoxigenin, and hapten, or any combination thereof.

17. A method for treating a PD-L1-related cancer and/or tumor, the method comprising contacting a PD-L1-related cancer and/or tumor with a treatment effective amount of the dimeric PD-L1 binding protein according to claim 1, whereby the PD-L1-related cancer and/or tumor is treated.

18. The method according to claim 17, wherein the dimeric PD-L1 binding protein according to claim 1 comprises a therapeutic moiety, optionally a therapeutic moiety selected from the group consisting of a monoclonal antibody or a fragment thereof, an extracellular domain of a receptor or a fragment thereof, a radionuclide, a cytotoxic compound, a cytokine, a chemokine, and an enzyme, or any combination thereof.

19. A method for treating an infectious disease, the method comprising administering to a subject in need thereof a treatment effective amount of the dimeric PD-L1 binding protein according to claim 1, whereby the infectious disease is treated.

20. The method according to claim 19, wherein the infectious disease is a chronic viral infection, optionally a chronic viral infection with human immunodeficiency virus (HIV), hepatitis B virus (HBV), or hepatitis C virus (HCV).
